(19) United States
(12) Patent Application Publication
(10) Pub. No.: US 2009/0076582 A1
(43) Pub. Date: Mar. 19, 2009

(54) PACKAGED BIOMEDICAL ELECTRODE UNIT AND METHOD OF INSPECTING QUALITY THEREOF

(75) Inventors: Yasuaki KOIKE, Tokyo (JP); Hajime Harada, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: NIHON KOHDEN CORPORATION

(21) Appl. No.: 12/269,332

(22) Filed: Nov. 12, 2008

Related U.S. Application Data
(62) Division of application No. 11/190,831, filed on Jul. 28, 2005.

(30) Foreign Application Priority Data
Jul. 28, 2004 (JP) P2004-219957

(51) Int. Cl. A61N 1/04

(52) U.S. Cl. 607/142

(57) ABSTRACT

Each of a pair of electrodes has a conductive face adapted to be brought into contact with a living body. A connector has a pair of terminals. Each of a pair of lead wires has a first end connected to one of the electrodes and a second end connected to one of the terminals. The electrodes, the connector and the lead wires constitute a biomedical electrode unit. A package body is adapted to hermetically packages the biomedical electrode unit in such a condition that the conductive faces of the electrodes are separatably adhered to each other, that the lead wires are coiled, and that the terminals are short-circuited by a conductive member.

### BACKGROUND OF THE INVENTION

The present invention relates to a package in which a biomedical electrode to be used as a defibrillation pad, or the like, is hermetically sealed so as to serve as a biomedical electrode unit. More particularly, the invention relates to a packaged biomedical electrode unit with which quality check of an electrode in the package can be performed conveniently and easily without opening the package, and, furthermore, the quality check of the electrode in the package can be performed accurately and quickly. The invention also relates to the method for inspecting the quality of the electrode.

Hermetically packaging a medical electronic apparatus such as, for instance, a medical electrode, for the purpose of storing the electrode in a stable condition is put into practice. In addition, periodic inspection with regard to electrical quality of the medical electrode under storage in such a hermetically packaged form without opening the package is put into practice.

For example, U.S. Pat. Nos. 5,402,884 and 5,579,919 disclose sealed packages for disposable medical electrodes, and are characterized in that electrical characteristics of the electrode in the sealed package can be periodically inspected without opening the sealed package.

Here, for inspecting electrical characteristics of an electrode in a sealed package, the electrode is connected to an electronic medical apparatus. For the purpose of attaining the connection, a portion of a lead wire which is connected to the electrode is extended to the outside of the sealed packaged in advance. In addition, a conductive wire or conductive piece for use in inspection of the electrode is additionally disposed, and one end of the conductive wire or conductive piece is caused to extend to the outside of the sealed package.

As described above, by hermetically packaging an electrode, safe and appropriate storage of the electrode and of a gel material coated on a surface of the electrode can be maintained. However, one end of each of the lead wires, respectively connected to the electrodes, and of the conductive wire or conductive piece for use in inspection is extended to the outside of the sealed package. Therefore, hermetic sealing of portions in the package through which the lead wires and the like are extended to the outside of the seated package, and interiors of the lead wires and the like must be secured. Accordingly, the configuration for hermetically sealing the package is complicated. In addition, since the lead wires and the like are extended to the outside of the sealed package, when strain is applied on the lead wires and the like during handling, or others, the hermetic sealing may be broken.

Japanese Patent Publication No. 2003-35626A discloses a sealed package in which a conductive content is wrapped by an insulative film. A pair of electrodes are brought into contact with the sealed package, or caused to adjacently oppose the same. A high DC voltage is applied between the electrodes, whereby presence/absence of a pinhole in the insulative film wrapping the content is detected on the basis of a spark generated between the pinhole and the electrode. Therefore, a disposable medical electrode or the like is inappropriate as a content of such a sealed package.

### SUMMARY OF THE INVENTION

It is therefore an object of the invention to provide a packaged biomedical electrode unit which hermetically packages a biomedical electrode unit, with which quality check of the biomedical electrode unit in the sealed package can be performed conveniently and easily without opening the package.

It is also an object of the invention to provide a method of inspecting quality of the packaged biomedical electrode which can be performed accurately and quickly.

In order to achieve the above objects, according to the invention, there is provided a packaged biomedical electrode unit, comprising:

a biomedical electrode unit, comprising:
a pair of electrodes, each of which has a conductive face adapted to be brought into contact with a living body;
a connector, having a pair of terminals;
a pair of lead wires, each of which has a first end connected to one of the electrodes and a second end connected to one of the terminals;

a conductive member; and

a package body, adapted to hermetically packages the biomedical electrode unit in such a condition that:
the conductive faces of the electrodes are separatably adhered to each other,
the lead wires are coiled; and
the terminals are short-circuited by the conductive member.

Preferably, the packaged biomedical electrode unit further comprises a separator having a plurality of small holes. The conductive face of each of the electrodes is provided with a conductive gel layer. The separator is interposed between the conductive faces of the electrodes so that the conductive gel layers come in contact with each other through the small holes.

With the above configurations, the entire biomedical electrode unit can be completely hermetically packaged conveniently and easily. Accordingly, progress in degradation of electrodes can be suppressed, and an effective storage period of the biomedical electrode unit can be extended in addition, accurate and quick quality check of the biomedical electrode unit in the sealed package can be performed easily and conveniently without opening the package.

According to the invention, there is also provided a method of inspecting quality of the above packaged biomedical electrode unit, comprising steps of:

providing a first coil and a second coil,

placing the packaged biomedical electrode unit such that the coiled lead wires are situated in the vicinity of the first coil and the second coil,

applying an AC voltage to the first coil to generate a magnetic field, so that an electromotive force is generated in the coiled lead wires by the magnetic field; and

detecting a voltage generated in the second coil by the electromotive force.

According to the invention, there is also provided a method of inspecting quality of the above packaged biomedical electrode unit, comprising steps of:

providing a resistor and a coil;

placing the packaged biomedical electrode unit such that the coiled lead wires are situated in the vicinity of the coil;

applying an AC voltage to the coil through the resistor to generate a magnetic field, so that an electromotive force is generated in the coiled lead wires by the magnetic field; and

detecting a voltage generated in the coil by the electromotive force.

In each of the above methods, it is determined that the biomedical electrode unit is available when the voltage generated in the second coil is no greater than a prescribed value.

With the above configurations, an inspection for determining quality of the packaged biomedical electrode unit can be performed conveniently and easily without opening the biomedical electrode unit in the package. In addition, the quality of the packaged biomedical electrode unit can be determined accurately and quickly.

### BRIEF DESCRIPTION OF THE DRAWINGS

The above objects and advantages of the present invention will become more apparent by describing in detail preferred exemplary embodiments thereof with reference to the accompanying drawings, wherein:

FIG. 1 is a section view of a main part of a packaged biomedical electrode unit according to a first embodiment of the invention;

FIG. 2 is a perspective view of a connector in the packaged biomedical electrode unit of FIG. 1;

FIG. 3 is a perspective view showing how a biomedical electrode unit is packaged;

FIG. 4 is a schematic perspective view showing an apparatus for inspecting quality of the packaged biomedical electrode unit of FIG. 1;

FIG. 5 is a graph showing a relationship between an internal resistance of a closed circuit formed by the biomedical electrode unit of FIG. 3 and a voltage detected in the apparatus of FIG. 4; and

FIG. 6 is a schematic perspective view showing an apparatus for inspecting quality of the packaged biomedical electrode unit, according to a second embodiment of the invention.

### DETAILED DESCRIPTION OF THE EMBODIMENTS

Embodiments of the invention will be described below in detail with reference to the accompanying drawings.

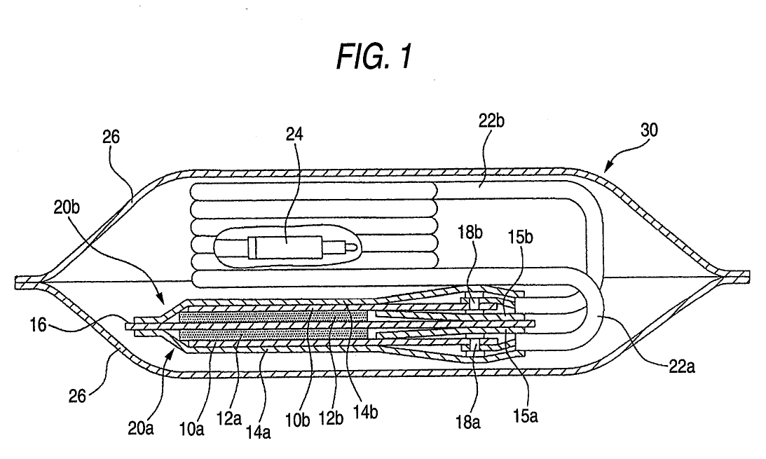

FIG. 1 shows a packaged biomedical electrode unit, for use in a defibrillator, according to a first embodiment of the present invention. Reference numerals 10a and 10b respectively denote flexible sheet electrodes. A conductive gel layer 12a is disposed on one side face of the flexible sheet electrode 10a, and a non-conductive member 14a is affixed on the other side of the same by an adhesive agent. A conductive gel layer 12b is disposed on one side face of the flexible sheet electrode 10b, and a non-conductive member 14b is affixed on the other side of the same by an adhesive agent. Thus, a pair of electrode members 20a and 20b are configured.

The thus-configured pair of electrode members 20a and 20b detachably adhere together the conductive gel layers 12a and 12b in a mutually-facing manner with a separator 16 therebetween. In this case, the separator 16 is configured so as to include a plurality of pores therein. Accordingly, the conductive gel layers 12a and 12b are partially brought into contact and electrical conduction, and can be separated easily at the time of usage of the biomedical electrode unit. One end of a lead wire 22a is electrically connected to one end of the flexible sheet electrode 10a by way of a crimp pin 18a, and one end of a lead wire 22b is electrically connected to one end of the flexible sheet electrode 10b by way of a crimp pin 18b. Meanwhile, reference numerals 15a and 15b denote non-conductive members which cover respective connecting sections between the flexible sheet electrodes 10a, 10b and the lead wires 22a, 22b.

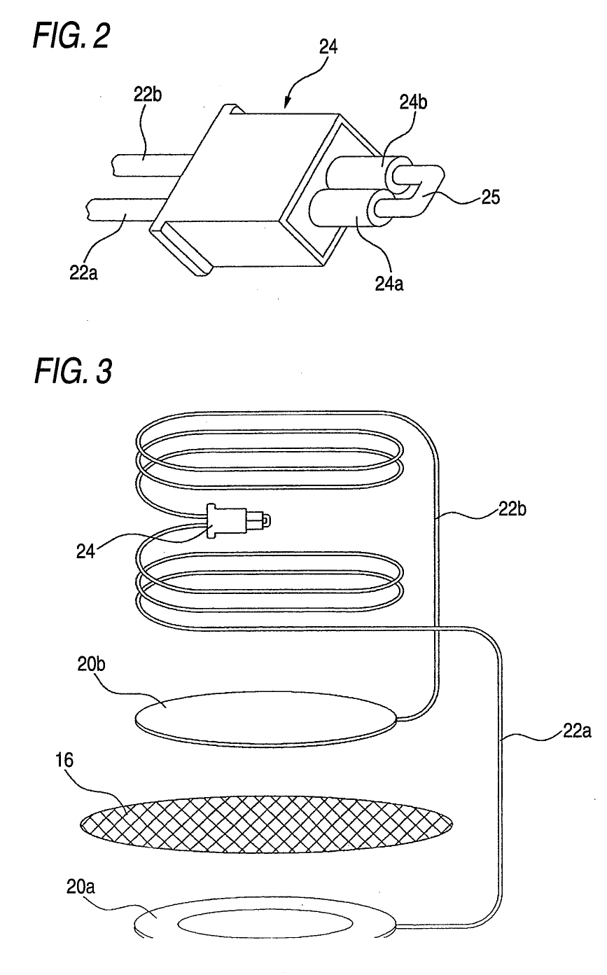

As shown in FIG. 2, one end of each of the lead wires 22a and 22b, extending from the other end of each of the electrode members 20a and 20b, and having a predetermined length, is connected to a connector 24 having terminal sections 24a and 24b. A short-circuit pin 25 form a short circuits between the terminal sections 24a and 24b, thereby constituting a single closed circuit in which the pair of electrode members 20a and 20b are brought into electrical conduction. The lead wires 22a and 22b are wound into a coil shape which can generate an electromotive force by an external magnetic field, and disposed on one side face of the adhered pair of electrode members 20a and 20b. The entire biomedical electrode unit is hermetically packaged by a non-magnetic and non-conductive hermetic packaging material 26, thereby constituting a packaged biomedical electrode unit 30. Meanwhile, as the hermetic packaging material 26, any of a variety of known packaging materials for hermetically packaging a variety of medical materials may be employed.

FIG. 3 shows a setting condition for packaging a biomedical electrode unit of the above-mentioned packaged biomedical electrode unit 30. More specifically, the pair of electrode members 20a and 20b are caused to oppose the conductive gel layers 12a and 12b and to adhere in a state of facing each other with the separator 16 therebetween. Subsequently, the lead wires 22a and 22b, which respectively extend from the electrode members 20a and 20b, are short-circuited by way of the short-circuit pin 25 at the terminal sections 24a and 24b of the connector 24. Thus, a single closed circuit including conduction through the pair of electrode members 20a and 20b is configured. The thus-short-circuited lead wires 22a and 22b are wound unidirectionally as shown in the drawing, and disposed on one side face of the adhered pair of electrode members 20a and 20b. The entire biomedical electrode unit having been set as above is hermetically packaged by the hermetic packaging material 26 as shown in FIG. 1.

Next, a method for inspecting quality of flexible sheet electrodes 10a, 10b, and the like, of a biomedical electrode unit configured as above will be described.

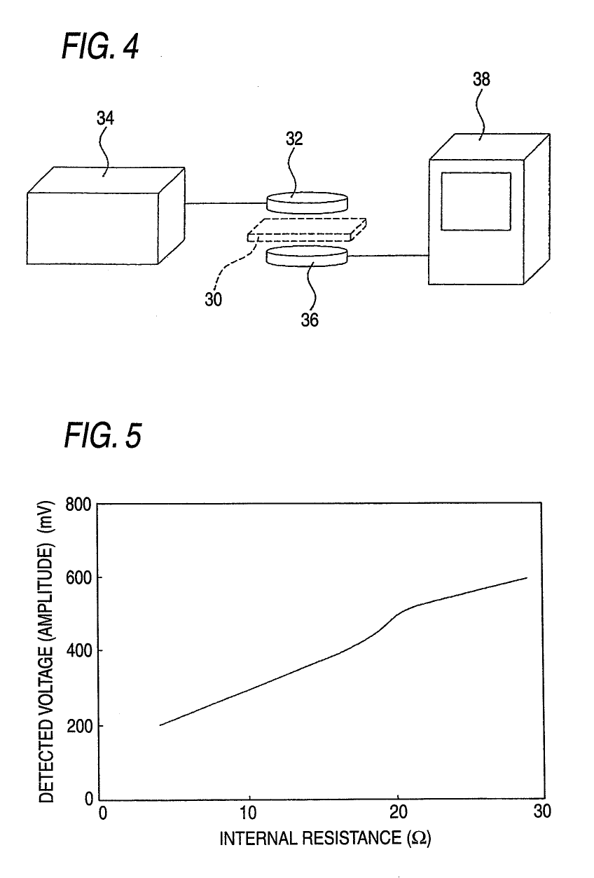

As shown in FIG. 4, the apparatus for implementing the inspection method is configured as follows. An oscillator coil 32 and a receiver coil 36 are disposed in mutual opposition, and also concentrically with the coil having been formed by winding the lead wires 22a and 22b unidirectionally. The oscillator coil 32 is disposed above the coil, and the receiver coil 36 is disposed below the same. The oscillator coil 32 is connected to an oscillator 34, and an AC voltage constituted of a predetermined frequency and voltage is applied thereto. The receiver coil 36 is connected to a voltage detector 38, such as an oscilloscope, thereby detecting a voltage (electromotive force) generated upon application of the voltage. When the flexible sheet electrodes 10a, 10b, or the like, are degraded, internal resistance therein increases. Accordingly, the voltage (electromotive force) generated in the receiver coil 36 increases. Therefore, when the above configuration is adopted, quality of the flexible sheet electrodes 10a, 10b, and the like, can be determined on the basis of the voltage (electromotive force) detected by the voltage (electromotive force) detector 38, such as the oscilloscope.

Next, an example of a voltage change generated in the receiver coil 36 in relation to a change in the internal resistance of the closed circuit which includes conduction through the pair of electrode members 20a and 20b will be described by reference to a specific example. Meanwhile, for the purpose of varying the internal resistance, measurement was conducted with a variable resistor inserted in the closed circuit in series connection.

The lead wires 22a and 22b were formed into a coil having a diameter of about 100 mm and ten turns. Each of the oscillator coil 32 and the receiver coil 36 was configured as a coil having a diameter of about 120 mm and twenty turns. The coils 32 and 36 were disposed in mutual opposition with a clearance of about 20 mm therebetween. The packaged biomedical electrode unit 30, having a thickness of about 10 mm, was inserted between the coils 32 and 36, and disposed in such a manner that the coil, having been formed by winding the lead wires 22a and 22b unidirectionally, was located concentrically with the coils 32 and 36. In addition, the oscillator coil 32, the receiver coil 36, and the packaged biomedical electrode unit 30 were respectively fixed by appropriate jigs, or the like, so as not to be displaced from their respective relative positions. Thus, a voltage having a frequency of 500 KHz and an output of 2 V (p-p) was applied to the oscillator coil 32 from the oscillator 34.

Under normal conditions, the single closed circuit wherein the pair of electrode members 20a and 20b are brought into electrical conduction in the biomedical electrode unit has an internal resistance of about 4Ω. However, when the flexible sheet electrode 10a, 10b, or the like, is degraded, as a result of degradation in electrical characteristics, the internal resistance increases. Here, the variable resistor having been inserted in the closed circuit in the biomedical electrode unit was varied within a range of 0 to 25Ω (4 to 29Ω in total internal resistance), and, thereupon, voltages (electromotive forces) detected by the voltage detector 38 such as an oscilloscope were measured. The measurement result reveals a characteristic such that, as shown in FIG. 5, the detected voltages increased (200 to 600 mV) as a result of the increase in the internal resistance.

Therefore, according to the embodiment, the following setting is applicable. That is, when a voltage value (electromotive force) detected by the voltage detector 38 is a predetermined reference value or lower, the flexible sheet electrodes 10a, 10b, and the like, are determined to be available. When the detected value exceeds the predetermined reference value, the flexible sheet electrodes 10a, 10b, and the like, are determined to be unavailable. The detection value varies depending on a type of the biomedical electrode unit and devices for implementing the inspection method, such as the oscillator coil and the receiver coil. Therefore, the reference value shall be set to an appropriate value.

In the above-mentioned embodiment of an apparatus configuration for implementing the inspection method for determining quality of the packaged biomedical electrode unit 30, the oscillator coil 32 and the receiver coil 36 are disposed concentrically, with the packaged biomedical electrode unit 30 disposed therebetween. However, the arrangement of the coils 32 and 36 is not limited thereto.

For instance, the oscillator coil 32 and the receiver coil 36 may be positioned so that the oscillator coil 32 and the receiver coil 36 are vertically adjacent to each other, and the adjacent coils 32 and 36 are disposed either adjacently above or adjacently below the packaged biomedical electrode unit 30. Also in this case, the coils 32 and 36, and the packaged biomedical electrode unit 30 are to be respectively fixed by appropriate jigs, or the like, so as not to be displaced from their respective relative positions.

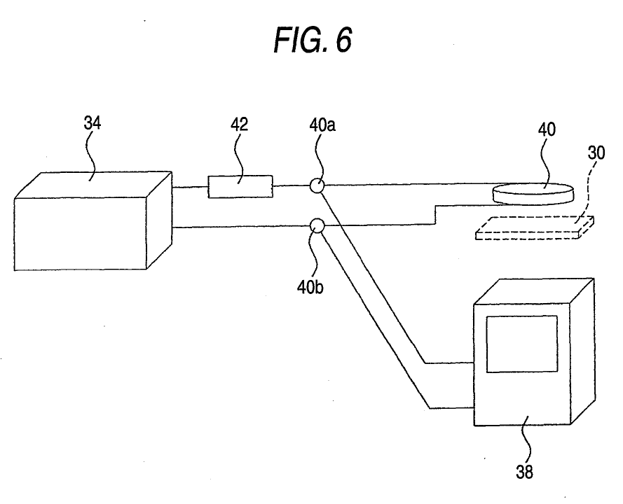

Next, a second embodiment of the apparatus configuration for implementing the inspection method will be described by reference to FIG. 6. The above-mentioned embodiment uses two coils constituted of the oscillator coil 32 and the receiver coil 36. However, the present embodiment uses a single coil. More specifically, in FIG. 6, a detector coil 40 is disposed so as to be adjacently above the packaged biomedical electrode unit 30 and so as not to displace its relative position in relation to the packaged biomedical electrode unit 30. A terminal 40a of the detector coil 40 is connected to the oscillator 34 by way of a resistor 42. A terminal 40b is directly connected to the oscillator 34. The oscillator 34 applies AC voltage of a predetermined frequency and voltage to the detector coil 40 by way of the resistor 42. In addition, the terminals 40a and 40b of the detector coil 40 are connected to the voltage detector 38, such as an oscilloscope, thereby detecting a voltage between the terminals 40a and 40b.

When such a configuration is adopted, in a case where the flexible sheet electrode 10a, 10b, or the like, is degraded, the internal resistance in the single closed circuit in which the pair of electrode members 20a and 20b are brought into electrical conduction increases as described above. Consequently, the terminal voltage of the detector coil 40 increases. Therefore, according to the present embodiment, quality of the flexible sheet electrodes 10a, 10b, and the like, can be determined on the basis of a detection signal output from the detector 38.

Although the present invention has been shown and described with reference to specific preferred embodiments, various changes and modifications will be apparent to those skilled in the art from the teachings herein. Such changes and modifications as are obvious are deemed to come within the spirit, scope and contemplation of the invention as defined in the appended claims.

1. A packaged biomedical electrode unit, comprising:
a biomedical electrode unit, comprising:
a pair of electrodes, each of which has a conductive face adapted to be brought into contact with a living body;
a connector, having a pair of terminals;
a pair of lead wires, each of which has a first end connected to one of the electrodes and a second end connected to one of the terminals;
a conductive member; and
a package body, adapted to hermetically packages the biomedical electrode unit in such a condition that:
the conductive faces of the electrodes are separatably adhered to each other;
the lead wires are coiled; and
the terminals are short-circuited by the conductive member.

2. The packaged biomedical electrode unit as set forth in claim 1, further comprising a separator having a plurality of small holes, wherein:
the conductive face of each of the electrodes is provided with a conductive gel layer; and
the separator is interposed between the conductive faces of the electrodes so that the conductive gel layers come in contact with each other through the small holes.

3-6. (canceled)
